(19) United States
(12) Patent Application Publication
(10) Pub. No.: US 2026/0069723 A1
(43) Pub. Date: Mar. 12, 2026

(54) LIPID MICROBUBBLE LYOPHILIZED POWDER COMPOSITION AND PREPARATION METHOD THEREOF

(71) Applicant: BEIJING QI-HUI BIOPHARMACEUTICAL CO., LTD., Beijing (CN)

(72) Inventors: Puchun FENG, Beijing (CN); Xiang LI, Beijing (CN); Suyun LIU, Beijing (CN)

(73) Assignee: BEIJING QI-HUI BIOPHARMACEUTICAL CO., LTD., Beijing (CN)

(21) Appl. No.: 19/107,948

(22) PCT Filed: Aug. 18, 2023
(86) PCT No.: PCT/CN2023/113783

§ 371 Date: Feb. 28, 2025

(30) Foreign Application Priority Data
Sep. 9, 2022 (CN) 202211099535.X

(51) Int. Cl. A61K 49/22; A61K 9/16; A61K 9/19

(52) U.S. Cl. CPC A61K 49/223; A61K 9/1617; A61K 9/1641; A61K 9/19

(57) ABSTRACT

Provided are a lipid microbubble lyophilized powder composition and a preparation method therefor. The composition comprises 1 part by weight of distearoylphosphatidylcholine, 1 part by weight of sodium 1,2-palmitoylphosphatidylglycerol, 100-240 parts by weight of polyethylene glycol 4000, and 0.1-0.3 parts by weight of palmitic acid. The lyophilized powder composition and a physiologically acceptable gas form a sealed vial. The lyophilized powder composition is used for ultrasonic contrast after reconstitution. By adjusting the proportion of each component of the lipid microbubble lyophilized powder composition and process parameters, the lipid microbubble lyophilized powder composition is made to have the advantages of a narrow particle size distribution of reconstituted microbubbles, high microbubble stability, being less likely to crack, good pressure resistance, etc. During ultrasonic contrast, the composition can achieve stronger image acoustic signals and stay longer at the target part.

### TECHNICAL FIELD

The present application belongs to the field of medicine, and relates to a lipid microbubble lyophilized powder composition and a preparation method thereof, in particular to an ultrasound contrast agent for improving the contrast of ultrasound images.

### BACKGROUND

Ultrasound contrast agent (UCA) is a kind of diagnostic drug that can cause strong scattering of blood after being injected into the peripheral vein, which can significantly enhance the ultrasonic medical detection signal. With the rapid development of ultrasound medicine and clinical pharmacology, contrast-enhanced ultrasound technology has become one of the fastest-growing technologies in the field of medical imageologyimaging today.

Sulphur hexafluoride microbubbles for injection (tradename: Sonovue®) is an ultrasound contrast agent that has been on the market for many years in China and other countries, used for contrast-enhanced ultrasound diagnosis, and is widely used in a variety of indications such as liver imaging, echocardiography, and urography.

However, with its clinical application, clinical researchers have found that there is room for improvement in Sonovue contrast agent, which can mainly be made in the following aspects: first of all, the stability of microbubbles after contrast agent reconstitution needs to be improved. In the using process of Sonovue, if the first contrast is not clear enough, the second contrast can be performed, but there is a problem that the enhancement effect of the second contrast is not as good as the first contrast, the reason is that in the contrast process of Sonovue after the reconstitution and preparation, the volume concentration of the microbubbles in the solution after the preparation gradually decreases over time, which is not conducive to contrast imaging. According to the literature (Influence of Bubble Size Distribution on the Echogenicity of Ultrasound Contrast Agents, Investigative Radiology, Volume 35, Number 11, 661-671), the particle size of ultrasound contrast agent microbubbles is correlated with the resonance frequency, and the higher the volume concentration of the central particle size within a certain range, the stronger its resonance frequency, i.e., the stronger the reflected acoustic signal. Therefore, it is an urgent problem to improve the stability of contrast agent microbubbles, thereby maintaining the volume concentration of microbubbles.

Furthermore, the contrast effect of the contrast agent can be enhanced by increasing the volume concentration of the contrast agent microbubbles.

In addition, the pressure resistance of microbubbles needs to be improved. According to the Chinese literature “Experimental study on the Influence of Pressure on the Backscattering Intensity of Contrast Agent Microbubbles” (paper, author: Min PAN, 2004 class of Huazhong University of Science and Technology, major in imaging medicine and nuclear medicine), Sonovue is suitable for ultrasound probes with a center frequency of 2-7.5 MHz. However, linear probes used in superficial organs such as thyroid and mammary gland generally have frequencies above 10 MHz. Under the high-frequency probe, the microbubbles of Sonovue are easy to rupture, resulting in a short residence time of the microbubbles in the thyroid, mammary gland and other organs, and the contrast effect is poor. Therefore, it is an urgent problem to improve the pressure resistance of microbubbles to expand its application scope.

Therefore, how to overcome the above-mentioned shortcomings of the prior art and improve the use effect of contrast agent is still an urgent problem to be solved in the field.

### SUMMARY

In order to solve the problems of poor stability of Sonovue microbubbles after reconstitution, the gradual decrease of the volume concentration of microbubbles over time, and the poor pressure resistance of microbubbles, the present application provides a lyophilized powder composition of lipid microbubble ultrasonic contrast agent, so as to obtain a contrast agent with good stability, high volume concentration of microbubbles, good acoustic response and good pressure resistance.

In a first aspect, the present application provides a lyophilized powder composition for the preparation of a lipid microbubble ultrasonic contrast agent, and the composition includes distearoylphosphatidylcholine (DSPC), 1,2-dipalmitoylphosphatidylglycerol sodium (DPPG-Na), poly(ethylene glycol) 4000 (PEG4000) and palmitic acid; wherein DSPC is 1 part by weight, DPPG-Na is 1 part by weight, PEG4000 is 150-200 parts by weight, and palmitic acid is 0.1-0.3 parts by weight.

Preferably, the PEG4000 is 170-185 parts by weight.

Preferably, the PEG4000 is 173-185 parts by weight.

In a second aspect, the present application provides a sealed vial containing the lyophilized powder composition as described in the first aspect, as well as a physiologically acceptable gas, for the preparation of a lipid microbubble ultrasound contrast agent.

In some embodiments, the gas may be a fluorinated gas, wherein the fluorinated gas may be any one or a combination of two of perfluoromethane, perfluoropropane, perfluorobutane, perfluoropentane and perfluorohalide.

In other embodiments, the gas may be sulfur hexafluoride.

In other embodiments, the gas may also be a low water-soluble inert gas; preferably, the low water-soluble inert gas can be nitrogen, argon, etc.

In a third aspect, the present application provides a preparation method for the sealed vial of lipid microbubble ultrasonic contrast agent, which includes the following steps:
(1) dissolving DSPC, DPPG-Na, PEG4000 and palmitic acid in a solvent to form a solution;
(2) filling the solution in a vial rapidly and performing vacuum lyophilizing to remove the solvent;
(3) introducing a physiologically acceptable gas to the vial and sealing the vial; and
(4) placing the sealed vial in an incubator for activation for 12-24 h.

In some embodiments, the solvent in step (1) is any one or a mixture of more of n-hexane, isopropanol, cyclohexanol, 2-methyl-2-butanol, tert-butanol or n-butanol.

Preferably, the solvent in step (1) is a mixture of tert-butanol and isopropanol, or a mixture of n-hexane and isopropanol.

In the process of research, the inventor found that the heating and activation of the lyophilized powder of lipid microbubble ultrasonic contrast agent is more conducive to forming a concentrated distribution of the particle size of the microbubbles after reconstitution, and the formed microbubbles have a higher volume concentration, better acoustic response, and also better pressure resistance.

Preferably, a temperature of the incubator in step (4) is 35-50° C., and more preferably the temperature of the incubator is 45° C.

Preferably, the activation in step (4) is performed for a period of 18-22 h.

In a fourth aspect, the present application provides a preparation method for a lipid microbubble ultrasonic contrast agent, which includes: under the existence of the physiologically acceptable gas, dispersing the lyophilized powder composition as described in the first aspect in normal saline (0.9% sodium chloride aqueous solution) to form a microbubble suspension, i.e., lipid microbubble ultrasonic contrast agent.

Preferably, a volume concentration of the microbubble suspension is more than or equal to 7 μL/mL; and more preferably, a volume concentration of the microbubble suspension is more than or equal to 8 μL/mL.

Preferably, a concentration of the microbubble suspension is more than or equal to 5×10⁸ microbubbles/cm³; and more preferably, a concentration of the microbubble suspension is more than or equal to 7×10⁸ microbubbles/cm³.

In a fifth aspect, the present application provides use of the lyophilized powder composition as described in the first aspect as a blood pool ultrasound contrast agent and a cavity ultrasound contrast agent.

In a sixth aspect, the present application provides a method of ultrasound imaging of the lipid microbubble ultrasonic contrast agent prepared from the method as described in the fourth aspect, which includes the following steps:
administering an effective amount of the lipid microbubble ultrasound contrast agent to a patient;
transmitting the ultrasound signal to a body part of the patient; and
collecting echogram signals from the body part.

Compared with the prior art, the present application has the following beneficial effects.

The lyophilized powder composition of the lipid microbubble ultrasonic contrast agent in the present application has many advantages, such as high stability of reconstituted microbubbles, good acoustic response, good pressure resistance, and the like. In addition, the image acoustic signal is stronger during contrast-enhanced ultrasound, the residence time in target site is longer, and the pressure resistance is better, which is suitable for a variety of organs and probe contrast modes.

### BRIEF DESCRIPTION OF DRAWINGS

FIG. 1 is the particle size distribution of the lipid microbubble ultrasound contrast agent of the batch 4-D samples in Example 4.

FIG. 2 is an ultrasonographic image of a rabbit kidney obtained from the 4-D sample after contrast-enhanced ultrasound in Example 8.

FIG. 3 is an ultrasonographic image of a rabbit kidney obtained from the Sonovue sample after contrast-enhanced ultrasound in Example 8.

FIG. 4 is an ultrasonographic image of a rabbit fallopian tube obtained from the 4-D sample after contrast-enhanced ultrasound in Example 8.

### DETAILED DESCRIPTION

### Definition and Abbreviations

DSPC: distearoylphosphatidylcholine
DPPG-Na: 1,2-dipalmitoylphosphatidylglycerol sodium
PEG4000: poly(ethylene glycol) 4000

### Explanation of Terms

Sonovue: generic name is sulphur hexafluoride microbubbles for injection; the drug is an ultrasound contrast agent developed by the Italian Bracco Imaging Group, and according to the instructions, each vial contains 59 mg of sulphur hexafluoride, 0.19 mg of distearoylphosphatidylcholine (DSPC), 0.19 mg of 1,2-dipalmitoylphosphatidylglycerol sodium (DPPG), 0.04 mg of palmitic acid, and 24.5 mg of poly(ethylene glycol) 4000.

Poly(ethylene glycol) 4000: Poly(ethylene glycol) is a polymer with the chemical formula HO(CH₂CH₂O)ₙH. Its properties vary depending on molecular weight, ranging from colorless, odorless and viscous liquid to waxy solid. PEG4000 is a poly(ethylene glycol) polymer with an average molecular weight of 4000, n=70-85, with a melting point of 53-56° C. The average molecular weight of poly(ethylene glycol) 4000 is about 4000 with the structure of

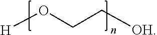

Microbubbles: Microbubbles are a class of pharmaceutical preparations, which generally consist of an air nucleus and a stabilized shell membrane. The coating material for bubbles is generally composed of lipids, polymers, or proteins, which have good biocompatibility and are safe for intravenous injection. Among them, phospholipids as the shell membrane of microbubbles are more flexible than the cross-linked polymer hard shell, and the membrane is easier to shrink, rupture, bend or re-expand under the action of ultrasound, thereby enhancing the sensitivity of microbubbles to sound waves.

Volume concentration of microbubbles: abbreviated as “volume concentration”, which refers to the ratio of the total volume of all microbubbles in the suspension to the total volume of the suspension, that is, the total volume concentration of microbubbles per unit volume of suspension (μL/mL).

Number of microbubbles: abbreviated as “number”, which refers to the ratio of the total number of microbubbles in the suspension to the total volume of the suspension, that is, the number of microbubbles per unit volume of suspension.

Reconstitution: refers to the process of adding normal saline to shake and dissolve the lyophilized powder preparation of ultrasonic contrast agent before clinical use to obtain a uniformly distributed microbubble suspension.

Remaining ratio: refers to the percentage of the volume concentration contained in the microbubble suspension, which is the microbubble suspension obtained after reconstitution and then subjected to room temperature or pressurized treatment during stability testing or pressure resistance testing, compared to the initial volume concentration before treatment.

Blood pool contrast-enhanced ultrasound: refers to the contrast-enhanced ultrasound imaging method in which the reconstituted ultrasound contrast agent microbubble suspension is injected into the systemic circulation through intravenous injection to obtain contrast-enhanced ultrasound imaging images of organs in vivo, including sonography of liver and abdominal organs, echocardiography, vascular Doppler, etc.

Cavity contrast-enhanced ultrasound: refers to the method of contrast-enhanced ultrasound imaging in which the reconstituted ultrasound contrast agent is injected into the uterus, gallbladder, bladder and other cavity organs through a syringe/catheter to obtain contrast-enhanced ultrasound imaging images of organs.

The present application is further described below in terms of specific embodiments. It should be clear that the embodiments should not be regarded as a specific limitation to the present application.

### Example 1 Preparation of a Lyophilized Composition by Lyophilized Directly

DSPC, DPPG, palmitic acid, and poly(ethylene glycol)-4000 were weighed and dissolved in an organic solvent. Then the solution was divided into vials in proportion (1:1: (0.1-0.3): (100-240)). After that, the vials were covered with bromobutyl rubber stoppers and semi-tucked, the vials were then pushed into the shelves of a lyophilizer and vacuum-lyophilized for 24 hours. Subsequently, the sulphur hexafluoride gas was flushed into the vials, and the stoppers were pressed and sealed to obtain a sealed lyophilized composition.

Preparation method of microbubbles: normal saline was injected into the vial before use, and the vial was shaken until the lyophilized powder was completely dispersed into a uniform white emulsion.

### Example 2 Preparation and Parameter Detection of a Lyophilized Composition with Different Dosage of PEG4000

Referring to the preparation method of Example 1, and different parts by weight of PEG4000 were used.

5 mL of 0.9% sodium chloride aqueous solution was injected for reconstitution before use, and part of the sulphur hexafluoride was encapsulated into milky-white suspension microbubbles. The number and volume concentration of the microbubbles were analyzed by a Multisizer 4e Coulter counter (Beckman Coulter, Inc.), and the results are shown in Table 1 below.

TABLE 1

| | PEG4000 | | Average | Volume |
|---|---|---|---|---|
| | parts by | Number | particle | concentration |
| Batch | weight | (10⁶/mL) | size (μm) | (μL/mL) |
| 1-A | 100 | 759 | 1.60 | 8.92 |
| 2-A | 120 | 951 | 1.58 | 9.68 |
| 3-A | 135 | 1212 | 1.60 | 11.13 |
| 4-A | 150 | 1434 | 1.62 | 12.24 |
| 5-A | 160 | 1384 | 1.59 | 11.94 |
| 6-A | 173 | 1917 | 1.57 | 13.62 |
| 7-A | 185 | 2361 | 1.465 | 13.17 |
| 8-A | 200 | 3088 | 1.414 | 12.35 |
| 9-A | 220 | 3507 | 1.211 | 9.90 |
| 10-A | 240 | 3145 | 1.136 | 4.56 |

The results show that with the increase of the dosage of PEG4000 in the prescription, the volume concentration of microbubbles also increases, but the volume concentration decreases after the dosage of PEG4000 increases to a certain amount (parts by weight). Since the quality-standard volume concentration range of Sonovue is 2-10 μL/mL, the prescriptions with a microbubble volume concentration above 10 μL/mL in the present application have better effects than Sonovue, i.e., batches 3-A, 4-A, 5-A, 6-A, 7-A, and 8-A. Therefore, the optimal volume concentration can be achieved with 135-200 parts by weight of PEG4000.

### Example 3 Selection of Optimal Activation Temperature

Referring to the preparation method of Example 1, 40 mg of DSPC, 40 mg of DPPG, 8 mg of palmitic acid and 6.90 g of poly(ethylene glycol) 4000 (the dosage was the dosage of the highest volume concentration in Example 2, 173 parts by weight, that is, the dosage of batch 6-A) were weighed, dissolved and subpackaged. After that, the vials were subjected to lyophilization and filled with sulphur hexafluoride gas to form a lyophilized composition sealed in the vials. The lyophilized composition was divided into 4 groups, heated at 35° C., 40° C., 45° C. and 50° C. for 24 h separately. The specific design is shown in Table 2 below:

TABLE 2

| | | Heating | Heating |
|---|---|---|---|
| | Batch | temperature | time |
| 1 | 6-D | 35° C. | 24 h |
| 2 | 7-D | 40° C. | |
| 3 | 8-D | 45° C. | |
| 4 | 9-D | 50° C. | |

Before the detection, 5 mL of 0.9% sodium chloride aqueous solution was injected for reconstitution, and part of the sulphur hexafluoride was encapsulated in milky white suspension microbubbles. The number and volume concentration of the microbubbles were analyzed by a Multisizer 4e Coulter counter (Beckman Coulter, Inc.), and the results are shown in Table 3 below.

TABLE 3

| | | | Average | Volume |
|---|---|---|---|---|
| Heating | | Number | particle | concentration |
| temperature | Batch | (10⁶/mL) | size (μm) | (μL/mL) |
| 35° C. | 6-D | 874 | 1.56 | 9.59 |
| 40° C. | 7-D | 1581 | 1.55 | 11.73 |
| 45° C. | 8-D | 1839 | 1.51 | 12.37 |
| 50° C. | 9-D | 1394 | 1.62 | 10.70 |

The results showed that the volume concentration was lowest under heat treatment at 35° C., and the volume concentration was highest under heat treatment at 45° C.

### Example 4 Selection of Optimal Activation Time

Referring to the preparation method of Example 1, 40 mg of DSPC, 40 mg of DPPG, 8 mg of palmitic acid and 6.90 g of poly(ethylene glycol) 4000 (the dosage corresponded to that of the highest volume concentration in Example 2) were weighed. The vials were filled with sulphur hexafluoride gas to form a lyophilized composition sealed in the vials. The vials were placed in an incubator for activation for 0 h, 6 h, 12 h, 24 h, and 48 h. Before the detection, 5 mL of 0.9% sodium chloride aqueous solution was injected for reconstitution, and part of the sulphur hexafluoride was encapsulated in milky white suspension microbubbles. The number and volume concentration of the microbubbles were analyzed by a Multisizer 4e Coulter counter (Beckman Coulter, Inc.), and the results are shown in Table 4 below.

TABLE 4

| | | | | |
|---|---|---|---|---|
| | | | Average | Volume |
| Heating | | Number | particle | concentration |
| time | Batch | (10⁶/mL) | size (μm) | (μL/mL) |

| | | | | | |
|---|---|---|---|---|---|
| 0 | h | 1-D | 643 | 1.25 | 4.50 |
| 6 | h | 2-D | 1759 | 1.47 | 11.72 |
| 12 | h | 3-D | 1658 | 1.58 | 12.39 |
| 24 | h | 4-D | 2014 | 1.53 | 12.32 |
| 48 | h | 5-D | 1700 | 1.56 | 12.20 |

The table shows that the volume concentration reaches its peak after more than 12 h of heat treatment. Subsequently, the volume concentration does not change substantially with the increase in heat treatment time. Based on the FDA application data of Sonovue (FDA, CENTER FOR DRUG EVALUATION AND RESEARCH, Lumason™, 203684Orig1s000, Chemistry Review(s)), the number of microbubbles per milliliter is (1.5-5.6)×10⁸, and the number of contrast agent microbubbles in all prescriptions in the above table are better than that of Sonovue.

Note: Lumason™ and Sonovue are tradenames of the same product listed in different countries.

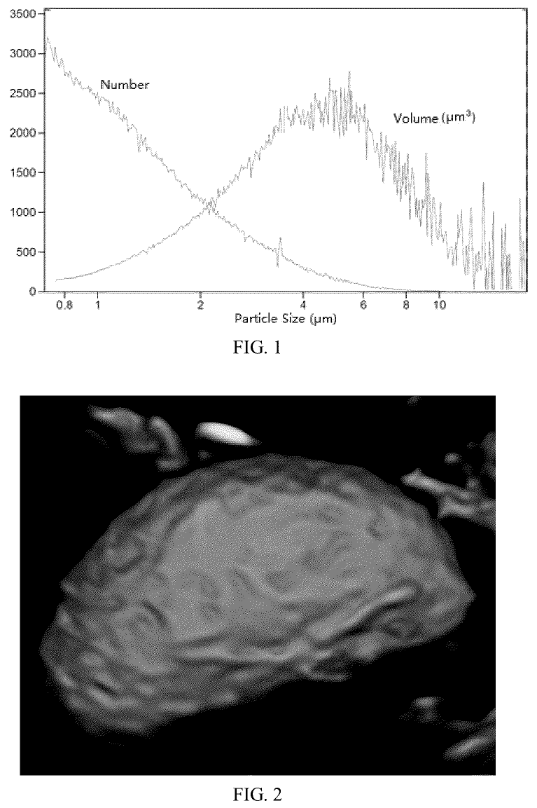

FIG. 1 shows the sampling test results of batch 4-D samples for 24 h.

In order to further explore the optimal process of heating for more than 12 h, the number and concentration of contrast agent microbubbles when heated for 12 h, 16 h, 18 h, 20 h and 22 h were further studied by using the above experimental scheme. The results are shown in Table 5 below.

TABLE 5

| | | | Average | Volume |
|---|---|---|---|---|
| Heating | | Number | particle | concentration |
| time | Batch | (10⁶/mL) | size (μm) | (μL/mL) |
| 12 h | 11-D | 1732 | 1.57 | 12.36 |
| 16 h | 12-D | 1768 | 1.56 | 12.18 |
| 18 h | 13-D | 1895 | 1.56 | 12.33 |
| 20 h | 14-D | 1953 | 1.54 | 12.31 |
| 22 h | 15-D | 2009 | 1.55 | 12.20 |

The above studies show that, the results of further heat treatment show that the volume concentration was basically unchanged with heat treatment time from 12 to 22 h.

### Example 5 Stability Comparison with Sonovue

Three commercially available Sonovue products were taken and the solution was prepared according to the instructions as the control group, and 3 samples of the batch 4-D in Example 4 were taken as the test group. The samples of the control group and the test group were taken respectively, and the volume concentration was taken as the parameter, and three samples were added to 5 mL of 0.9% sodium chloride aqueous solution to dissolve, and a Multisizer 4e Coulter counter (Beckman Coulter, Inc.) was used for analysis, the volume concentration of 0 min was recorded, and the particle size distribution of the microbubbles was detected again after standing at room temperature and atmospheric pressure for 10 min, 30 min and 60 min, respectively, and the results are shown in Table 6 below.

TABLE 6

| | | | Volume concentration (μL/mL) | | |
|---|---|---|---|---|---|
| | | Time | | Volume | Remaining |
| | Prescription | (min) | 0 min | concentration | ratio |
| 1 | Test group | 10 | 14.98 | 14.05 | 93.79 |
| 2 | | 30 | 13.75 | 12.80 | 93.09 |
| 3 | | 60 | 15.68 | 13.63 | 86.93 |
| 4 | Control | 10 | 7.796 | 7.354 | 94.33 |
| 5 | group | 30 | 7.705 | 5.365 | 69.63 |
| 6 | | 60 | 7.0878 | 4.831 | 68.16 |

It can be seen from the above table that the stability of the test group was significantly better than that of the control group. There was no significant difference between the two at 10 min, and the remaining proportion of volume concentration was more than 90%. After 60 min, the volume concentration of the test group remained 86.93%, but that of the control group decreased significantly, only remaining 68.16%. The remaining proportion of volume concentration in the test group was about 20% higher than that of the control group at 60 min.

Furthermore, three samples of the batch 7-D in Example 3 were taken as test group 2, and the microbubble stability test was carried out again by using the above scheme, and the test results are shown in Table 7 below.

TABLE 7

| | | | Volume concentration (μL/mL) | | |
|---|---|---|---|---|---|
| | | Time | | Volume | Remaining |
| | Prescription | (min) | 0 min | concentration | ratio |
| 1 | Test group 2 | 10 | 11.98 | 11.21 | 93.57 |
| 2 | | 30 | 11.26 | 10.37 | 92.10 |
| 3 | | 60 | 12.35 | 10.32 | 83.56 |

The results showed that the stability of test group 2 was substantially the same as that of the test group, which was significantly better than that of the control group. There was no difference between the three at 10 min, and the remaining proportion of volume concentration was more than 90%. The volume concentration of the test group and the test group 2 decreased to 86.93% and 83.56% respectively after 60 min, but the control group only remained 68.16%.

It can be seen that the stability of the microbubbles prepared by the scheme of Example 4 was better than that of Sonovue of the control group.

### Example 6 Research on the Pressure Resistance Performance of Microbubbles

Pressure test method: Using a self-made simple pressurization device, air was pushed into a filled and sealed vial for pressurization by a syringe. The sample was exposed to overpressures of 100 mmHg, 120 mmHg, 140 mmHg, and 180 mmHg for 90 s. Then, the overpressures were released, and the concentration and average particle size of the microbubbles were measured respectively. (Pressurization device: one end of the mercury sphygmomanometer was connected to a cork-sealed jar containing a small vial with a rubber tube. The vial contained the test contrast agent, and air was pushed into it through a plug by a syringe for pressurization. The air pressure inside the vial was indicated by the scale of the mercury sphygmomanometer.)

Four Sonovue products were taken and the solution was prepared according to the instructions as the control group, and four samples of the batch 4-D in Example 4 were taken as the test group. The samples of the control group and the test group were taken respectively, the volume concentration was taken as the parameter, and four samples were taken and added to 5 mL of 0.9% sodium chloride aqueous solution to reconstitution, and a Multisizer 4e Coulter counter (Beckman Coulter, Inc.) was used for analysis. The volume concentration of 0 min was recorded, and the samples were placed at the pressure of 120 mmHg for 1 min, 3 min, 5 min and 10 min, respectively, and the particle size distribution of the microbubbles was detected again after restored to normal pressure and shaken evenly, and the results are shown in Table 8 below.

TABLE 8

| | | | Volume concentration (μL/mL) | | |
|---|---|---|---|---|---|
| | | Time | | Volume | Remaining |
| | Prescription | (min) | 0 min | concentration | ratio |
| 1 | Test group | 1 | 14.12 | 12.38 | 87.68 |
| 2 | | 3 | 14.70 | 12.87 | 87.55 |
| 3 | | 5 | 12.55 | 9.170 | 73.07 |
| 4 | | 10 | 15.48 | 10.09 | 65.18 |
| 5 | Control | 1 | 7.183 | 5.413 | 75.36 |
| 6 | group | 3 | 7.364 | 4.763 | 64.68 |
| 7 | | 5 | 7.424 | 4.563 | 61.46 |
| 8 | | 10 | 7.412 | 3.629 | 48.96 |

The results showed a decreasing trend both in the test group and the control group with the extension of time within 10 min. After 10 min of 120 mmHg pressure test, 65.18% of the test group and 48.96% of the control group remained, indicating that the pressure resistance of the test group was better than that of the control group.

Using the above scheme, but with a difference that the heating temperature adopted was 40° C. and the heating time was 24 h. Taking four samples of the batch 7-D in Example 3 as the test group 2, the pressure resistance test of microbubbles was carried out again, and the results are shown in Table 9 below.

TABLE 9

| | | | Volume concentration (μL/mL) | | |
|---|---|---|---|---|---|
| | | Time | | Volume | Remaining |
| | Prescription | (min) | 0 min | concentration | ratio |
| 1 | Test group 2 | 1 | 12.26 | 10.78 | 87.93 |
| 2 | | 3 | 12.09 | 10.54 | 87.18 |
| 3 | | 5 | 11.83 | 8.58 | 72.53 |
| 4 | | 10 | 12.35 | 8.19 | 66.32 |

The results showed that the volume concentration of microbubbles in all groups decreased with the extension of time within 10 min, and more than 60% of the test group and test group 2 and 48.96% of the control group remained after 10 min of 120 mmHg pressure test, indicating that the pressure resistance performance of the test group and test group 2 was better than that of the control group.

It can be seen that the pressure resistance of the microbubbles prepared by the scheme of Example 4 was better than that of Sonovue of the control group.

### Example 7 Research on the Effect of Contrast-Enhanced Ultrasound

Test animals: female New Zealand white rabbits, weighing2-2.5 kg, 4˜5 months old, in good health.

Equipment model: GE E8 endocavitary probe RIC5-9-D, MI: 0.19, frequency: 4-9 MHz, used 3D real-time scanning to monitor the perfusion condition of contrast agent.

Samples for the test group: 1 sample from batch 4-D, injected with 5 ml of normal saline, shaken and reconstituted to obtain a homogeneous and white microbubble suspension.

Samples for the control group: 1 Sonovue, injected with 5 mL of normal saline, shaken and reconstituted to obtain a homogeneous white microbubble suspension.

### Result of Blood Pool Imaging

Contrast imaging of rabbit kidney: 0.1 mL of contrast agent was taken from the test group and the control group respectively, and injected into the ear marginal vein of the rabbits. The effect of kidney image development was detected according to the above mode. Evaluation was performed using image recording data after the injection of the contrast agent until the enhancement effect completely disappeared. The pictures were analyzed by Image J software. The various indicators were observed and compared.

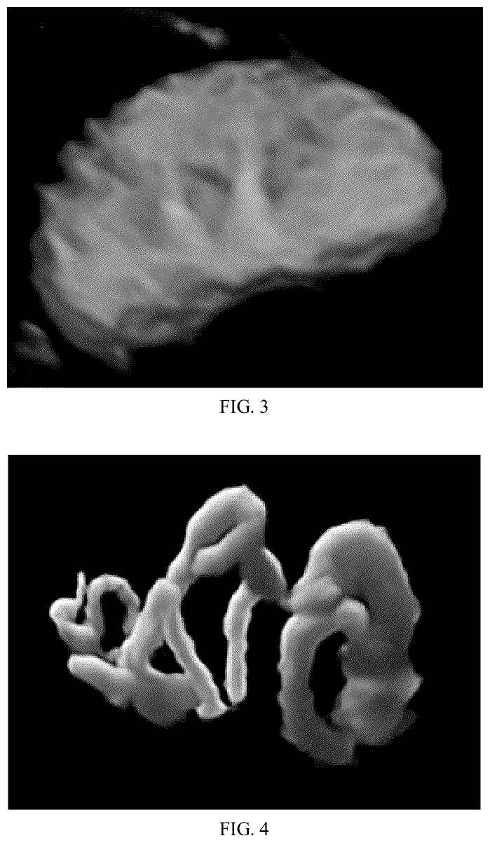

The representative picture of images obtained in the test group is shown in FIG. 2, and that obtained in the control group is shown in FIG. 3. Both of which can well show the characteristics of rabbit kidney. It can be seen from the comparison that the image in FIG. 2 has high brightness, full image, uniform development, and complete and smooth edge contour. FIG. 3 has slightly lower brightness, poor uniformity, and incomplete edge profiles.

### Result of Cavity Imaging

Contrast imaging of rabbit fallopian tube: 0.5 mL of the microbubble suspension of the test group was taken and diluted to 20 mL. The solution was slowly injected into the uterine cavity of the female rabbit through a contrast catheter, and the image was obtained through abdominal ultrasound scanning as shown in FIG. 4. The image has high brightness, with the whole uterus exhibiting homogeneous visualization, and the contour edge is clear and regular, which could show the normal physiological curved structure of the rabbit uterus.

Therefore, the contrast effect of the microbubbles prepared by the scheme of Example 4 was better than that of Sonovue of the control group.

In summary, it can be seen that the lipid microbubble suspension prepared by using the lipid contrast agent lyophilized powder composition in the present application has a high volume concentration after reconstitution, good pressure resistance of microbubbles, good stability, and a better contrast effect of microbubbles.

Finally, it should be noted that the above descriptions are only the preferred embodiments of the present application. They are merely used to illustrate the technical solutions of the present application and are not intended to limit its protection scope. Any modifications, equivalent substitutions, improvements, etc., made within the spirit and principles of the present application shall fall within its protection scope.

1. A lyophilized powder composition for the preparation of a lipid microbubble ultrasonic contrast agent, comprising distearoylphosphatidylcholine, 1,2-dipalmitoylphosphatidylglycerol sodium, poly(ethylene glycol) 4000 and palmitic acid;
wherein distearoylphosphatidylcholine is 1 part by weight, 1,2-dipalmitoylphosphatidylglycerol sodium is 1 part by weight, poly(ethylene glycol) 4000 is 150-200 parts by weight, and palmitic acid is 0.1-0.3 parts by weight.

2. The lyophilized powder composition according to claim 1, wherein poly(ethylene glycol) 4000 is 170-185 parts by weight.

3. The lyophilized powder composition according to claim 1, wherein poly(ethylene glycol) 4000 is 173-185 parts by weight.

4. A sealed vial containing the lyophilized powder composition according to of claim 1, as well as a physiologically acceptable gas, for the preparation of a lipid microbubble ultrasound contrast agent.

5. The sealed vial according to claim 4, wherein the gas may be a fluorinated gas; and
the fluorinated gas is any one or a combination of two of perfluoromethane, perfluoropropane, perfluorobutane, perfluoropentane and perfluorohalide.

6. The sealed vial according to claim 4, wherein the gas is sulfur hexafluoride.

7. The sealed vial according to claim 4, wherein the gas is a low water-soluble inert gas;
preferably, the low water-soluble inert gas is nitrogen or argon.

8. A method for preparing the sealed vial according to claim 4, comprising the following steps:
(1) dissolving distearoylphosphatidylcholine, 1,2-dipalmitoylphosphatidylglycerol sodium, poly(ethylene glycol) 4000 and palmitic acid in a solvent to form a solution;
(2) filling the solution in a vial rapidly and performing vacuum lyophilizing to remove the solvent;
(3) introducing a physiologically acceptable gas to the vial and sealing the vial; and
(4) placing the sealed vial in an incubator for activation for 12-24 h.

9. The method according to claim 8, wherein in step (1), the solvent is any one or a mixture of more of n-hexane, isopropanol, cyclohexanol, 2-methyl-2-butanol, tert-butanol or n-butanol.

10. The method according to claim 8, wherein in step (1), the solvent is a mixture of tert-butanol and isopropanol, or a mixture of n-hexane and isopropanol.

11. The method according to claim 8, wherein a temperature of the incubator is 35-50° C., preferably the temperature of the incubator is 45° C.

12. The method according to claim 8, wherein the activation in step (4) is performed for a period of 18-22 h.

13. A method for preparing a lipid microbubble ultrasonic contrast agent, comprising: under the existence of the physiologically acceptable gas, dispersing the lyophilized powder composition according to claim 1 in normal saline to form a microbubble suspension.

14. The method according to claim 13, wherein a volume concentration of the microbubble suspension is more than or equal to 7 μL/mL.

15. The method according to claim 14, wherein a volume concentration of the microbubble suspension is more than or equal to 8 μL/mL.

16. The method according to claim 13, wherein a concentration of the microbubble suspension is more than or equal to 5×10⁸ microbubbles/cm³.

17. The method according to claim 16, wherein a concentration of the microbubble suspension is more than or equal to 7×10⁸ microbubbles/cm³.

18. (canceled)

19. A method of ultrasound imaging, comprising the following steps:
administering an effective amount of the lipid microbubble ultrasound contrast agent prepared by the method according to claim 14 to a patient;
transmitting the ultrasound signal to a body part of the patient; and
collecting echogram signals from the body part.

20. (canceled)

21. A reconstituted lipid ultrasonic microbubble with increased volume concentration, improved stability, and/or improved acoustic response signal and pressure resistance, comprising the lyophilized powder composition according to claim 1.

22. A method for blood pool imaging or cavity imaging, comprising using the lyophilized powder composition according to claim 1.
